(19) United States
(12) Patent Application Publication
(10) Pub. No.: US 2009/0261275 A1
(43) Pub. Date: Oct. 22, 2009

(54) Particle therapy system, method for determining control parameters of such a therapy system, radiation therapy planning device and irradiation method

(76) Inventor: Eike Rietzel, Darmstadt (DE)

Correspondence Address: BRINKS HOFER GILSON & LIONE, P.O. BOX 10395, CHICAGO, IL, 60610, US

(21) Appl. No.: 11/989,267

(22) PCT Filed: Jul. 25, 2006
(86) PCT No.: PCT/EP2006/064645

§ 371 Date: Jan. 17, 2008

Related U.S. Application Data
(60) Provisional application No. 60/702,378, filed on Jul. 26, 2005.

(30) Foreign Application Priority Data
Jul. 26, 2005 (DE) 10 2005 034 912.9

(51) Int. Cl. A61N 5/00

(52) U.S. Cl. 250/492.1

(57) ABSTRACT

A method for determining control parameters of a therapy system for an irradiation sequence of a target volume to be irradiated from an irradiation direction is provided. The method includes automatically splitting up the target volume into a number of subvolumes, each of the subvolumes being no greater than the maximum scanning volume, and each of the volume elements being comprised in at least one subvolume, automatically determining a patient position and/or patient holder position as a first control parameter in which one of the subvolumes is arranged in the scanning area, and automatically determining a particle “sub” number for each volume element of a subvolume as a second control parameter, such that the sum of all the particle “sub” numbers of a first volume element corresponds to the required particle number of the first volume element.

The present patent document is a 35 U.S.C. § 371 application of PCT Application Ser. No. PCT/EP2006/064645 filed Jul. 25, 2006, designating the United States, which is hereby incorporated by reference. This patent document also claims the benefit of German patent application 10 2005 034 912.9 filed Jul. 26, 2007, which is hereby incorporated by reference.

### BACKGROUND

The present embodiments relate to a particle therapy system. The present embodiments further relate to the planning and carrying out of an irradiation with such a system, and to a radiation therapy planning device.

A particle therapy system usually has an accelerator unit and a high-energy beam guidance system. The acceleration of the particles, e.g. protons, carbon or oxygen ions, is performed, for example, with the aid of a synchrotron or a cyclotron.

The high-energy beam transport system guides the particles from the accelerator unit to one or more treatment stations. A distinction is made between fixed beam treatment stations in which the particles strike the treatment area from a fixed direction, and gantry-based treatment stations. In gantry-based treatment stations, it is possible to direct the particle beam onto the patient from various directions.

There are different radiation techniques, such as scanning techniques and scattering techniques, for irradiating a patient. Scattering techniques use of a large-area beam adapted to the dimensions of the volume to be irradiated. Scanning techniques scan a pencil beam with a diameter of a few millimeters to centimeters over the volume to be irradiated. When a scanning system is designed as a raster scanning system, the particle beam is directed pointwise onto a volume element of the raster until a previously defined particle number is applied. All the volume elements in the scanning area are irradiated one after another, preferably with overlapping pencil beams. The particle numbers for a volume element make a contribution to the dose not only in this volume element, but they contribute to the dose along the entire particle path.

A control and safety system of the particle therapy system ensures that in each case a particle beam characterized by the requested parameters is led into the appropriate treatment station. The parameters are defined in a treatment plan or a therapy plan. The therapy plan specifies how many particles, from which direction and with what energy, hit the patient or the volume elements. The energy of the particles determines the depth to which the particles penetrate into the patient. For example, the site of occurrence of the maximum in the interaction with the tissue during the particle therapy is the site at which the maximum of the dose is deposited. During treatment, the maximum of the deposited dose is located inside the tumor (or in the respective target zone in the case of other medical applications of the particle beam). Furthermore, the control and safety system controls a positioning device with the aid of which the patient is positioned with reference to the particle beam.

Particle therapy systems having a scanning system are disclosed, for example, in EP 0 986 070 or in “The 200-MeV proton therapy project at the Paul Scherrer Institute: Conceptual design and practical realization”, E. Pedroni et al., Med. Phys. 22, 37-53 (1995).

When planning a treatment, usually a number of irradiation fields having various incidence angles are planed individually. Each irradiation field is adjusted to the scanning system. In other words, when planning, fields whose dimensions are limited by a scanning area of the scanning system are individually planned in each case. The scanning area is given by the maximum deflection of the particle beam. A distinction is made here between 2D scanning (the deflection of the particle beam takes place in two directions) and 1D scanning. In 1D scanning, the patient is also moved stepwise in order to be able to irradiate in the second dimension as well.

There is a problem in irradiating a volume that is greater than a maximum scanning volume determined by the scanning area of the scanning system of the therapy system. An example of this is the treatment of a cancerous disease of the spine. With a length of, for example, 60 cm, the spine cannot be irradiated in one irradiation sequence when use is made of a scanning device with a scanning area of, for example, 40 cm×40 cm. In order to solve such a problem, it is proposed, for example, in “The 200-MeV proton therapy project at the Paul Scherrer Institute: Conceptual design and practical realization” to plan two fields that overlap one another, the doses of the individual fields adding together in the overlapping area. The patient is moved by the requisite distance between the irradiation of the two fields. Usually, this field patching necessitates renewed checking of the position of the patient relative to the scanning system in order to avoid faulty positioning.

### SUMMARY

The present embodiments may obviate one or more drawbacks or limiations inherent in the related art. For example, in one embodiment, the planning and carrying out of an irradiation of a volume that is greater than a maximum scanning volume determined by the scanning area of the scanning system of the therapy system are simplified. In another example, devices may simplify the planning and/or the irradiation.

In one embodiment, control parameters of a therapy system are determined that characterize an irradiation sequence in which a volume to be irradiated is irradiated from one, in other words, from substantially the same, irradiation direction. The irradiation sequence is a temporarily terminated unit of the irradiation. Such an irradiation sequence is preceded, for example, by an alignment and verification of the position of a patient who is, for example, positioned on a patient holding device of a positioning device of the therapy system. The verification of the position is then followed by the irradiation of the volume from a fixed irradiation direction.

The starting point of the method for determining control parameters is that the volume is subdivided into a multiplicity of volume elements, and that each volume element has been assigned a particle number to be applied that may produce the success of the therapy. The volume is greater than the maximum scanning volume of the scanning system. Such an encompassing dose distribution is not carried out in state of the art therapy planning procedures, since the particle numbers of volume elements that are to be applied are usually planned only for one irradiation field in each case. The dimensions of the volume irradiated with the aid of the irradiation field may be given by the scanning area.

The method for determining control parameters relates to a target volume to be irradiated that is greater than a maximum scanning volume determined by a scanning area of a scanning system of the therapy system. The volume to be irradiated is split up into a number of subvolumes, each of the subvolumes are no greater than the maximum scanning volume, and each of the volume elements are contained in at least one subvolume. Such a splitting up ensures that each volume element is irradiated in the irradiation sequence. Volume elements can be irradiated several times when they belong to a number of subvolumes. This is the case when subvolumes overlap one another.

Starting from the splitting up into subvolumes, a patient position and/or patient holder position is determined in which one of the subvolumes is arranged in the scanning area. In order to be able to irradiate the entire volume to be irradiated, such a control parameter is required for each subvolume. It is also sufficient to determine, in addition to one absolute position of one subvolume, relative positions of the remaining subvolumes starting from the known absolute position of the subvolume.

Moreover, a particle “sub” number is determined for each volume element of a subvolume. The particle “sub” number serves as a control parameter for the therapy system. If all the subvolumes are irradiated in accordance with the particle “sub” number, a condition for the particle “sub” number is that the sum of all the particle “sub” numbers of a volume element corresponds to the required particle number of this volume element.

Once a dose distribution over the volume to be irradiated has been planned, a user can automatically convert this dose distribution into an irradiation sequence that permits the target volume to be irradiated with a smaller scanning volume. The complicated planning of a number of irradiation fields is eliminated and the user gains time.

In one embodiment, the user specifies the position of a first subvolume with reference to the volume, for example, by arranging a first one of the subvolumes in the volume. The user may prescribe a size of an overlapping area between subvolumes. For example, the overlapping area may be displayed on a display unit. This further enables the user to subsequently check the arrangement and size of the overlapping areas and, if appropriate, to correct them. The position of the subvolumes and/or the particle “sub” number distributions may be displayed on a display unit The display enables the user to make a visual check of the result of the splitting up and of the control parameters associated therewith.

The splitting up of particle “sub” numbers of a volume element for two or more subvolumes may be provided in the overlapping area. For example, a gradient of a “dose ramp”, that is to say a particle “sub” number ramp, may be provided in the overlapping area.

A radiation therapy planning device for carrying out such a method includes a device for automatically splitting up the volume to be irradiated into a number of subvolumes, a device for automatically determining control parameters for positioning the subvolumes in the scanning area of the scanning system, and a device for automatically determining particle “sub” numbers for each volume element of a subvolume.

In one embodiment, for example, the irradiation method for irradiating a patient with high-energy particles from a therapy system has an irradiation sequence that is based on subvolumes, each of the subvolumes being no greater than the maximum scanning volume, and each of the volume elements being contained in at least one subvolume. The irradiation sequence is preceded by the patient adopting an irradiation position. The irradiation position may be, for example, on a patient holding device of a positioning device of the therapy system. The patient holding device may be, for example, a patient chair or a patient couch. The patient is preferably fixed in this irradiation position, for example, sitting, lying, or standing, and the position is verified by an imaging device.

For the radiation, the subvolumes are positioned in the scanning area one after the other. Volume elements arranged next to one another are thereby irradiated with the aid of particle “sub” numbers inside the scanning area by driving the scanning system in such a way that the sum of all the particle “sub” numbers of a volume element corresponds to the previously planned particle number.

The irradiation of a volume that is greater than a maximum scanning volume, which is determined by a scanning area of a scanning system, can be carried out automatically without further interventions of a user. For example, the irradiation and change in the patient's position are carried out automatically in the required sequence. If appropriate, the operator may be required to give clearance for a larger displacement. Inaccuracies in the positioning of the patient are minimized on the basis of the short temporal sequence of the irradiations of the subvolumes, and so the position of the patient is verified once before the irradiation sequence.

The impact of possible changes in the position of the patient on the applied dose distribution may be minimized because, in the overlapping area, the distribution of the particle “sub” numbers drops to the edge of the subvolume in the shape of a ramp. Alternatively, irradiation sequences can, for example, be planned for various days with differently arranged subvolumes such that any dose fluctuations owing to incorrect positionings are varied in three dimensions. A precondition for the overlapping of subvolumes and for the controlled superposition of doses in the overlapping area is the availability of a scanning system with the aid of which the position of a particle beam may be set in two dimensions in the region of a scanning area such that the doses acting can be accumulated on the plane by volume elements.

In one embodiment, a particle therapy system for irradiating a target volume of a patient that is to be irradiated includes a scanning system that can seta position of a particle beam in two dimensions in the region of a scanning area, a positioning device for positioning the volume of the patient that is to be irradiated relative to the scanning system, and a control unit for driving the scanning system and the positioning device. The particle therapy system carries out an irradiation where subvolumes are positioned in the scanning area one after the other and are irradiated from one and the same irradiation direction. The control unit is designed for processing control parameters that enable the subvolumes to be positioned in the scanning area of the scanning system and enable the irradiation of a volume element of the subvolume with a particle “sub” number in such a way that the sum of all the particle “sub” numbers of a volume element corresponds to a planned particle number of this volume element.

### BRIEF DESCRIPTION OF THE DRAWINGS

Further advantageous, features and details of the present embodiments will become evident from the description of illustrated exemplary embodiments given herein and the accompanying drawings, which are given by way of illustration only, wherein:

FIG. 1 shows a schematic view of one embodiment of a particle therapy system,

FIG. 2 shows a flowchart for an irradiation sequence, and

FIG. 3 shows a block diagram illustrating the splitting up into subvolumes of a volume to be irradiated.

### DETAILED DESCRIPTION

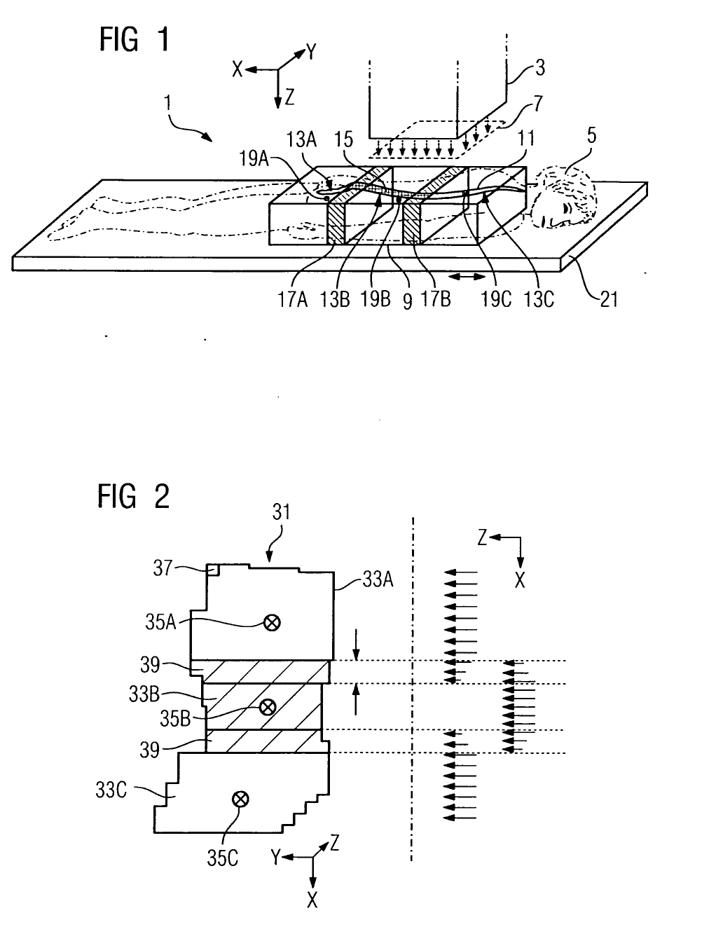

FIG. 1 shows irradiation location 1 of a particle therapy system. A scanning system 3 and a patient 5 lying thereunder are indicated schematically. The irradiation location 1 is part of a particle therapy system having an accelerator system and a high-energy beam guidance, in which particles are accelerated to energies of up to a few 100 MeV. The particles may be ions, such as protons or carbon ions. The scanning system 3 may be used to set the position of the beam in a parallel fashion in a scanning area 7. This scanning area has a size of 40 cm×40 cm, for example. The scanning area delimits a maximum scanning volume 9 in the X-Y plane (with the patient being unmoved). The extent of the scanning volume 9 in the Z-direction is a function of the energy of the particles.

By way of example, the aim in FIG. 1 is to irradiate a spine 11 of the patient 5. In other words, the volume to be irradiated is greater than a maximum scanning volume 9 determined by the scanning area 7. The term “greater” is to be understood in the sense that the dimensions of the volume to be irradiated are greater in at least one direction than the dimensions of the scanning volume, such that the volume to be irradiated does not fit into the scanning volume 9.

The irradiation of the volume to be irradiated, such as the spine 11 in FIG. 1, is performed in an irradiation sequence in which three subvolumes 13A, 13B, 13C are irradiated. Volume elements 15 are depicted in the subvolume 13B by way of illustration.

During therapy planning, particle numbers are determined for all the volume elements 15 of the volume to be irradiated. The determination is performed such that a planned dose distribution is effected. In other words, the desired dose is applied in each volume element in the case of an irradiation of all the volume elements 15 in the Z-direction.

The volume to be irradiated is split up into three subvolumes 13A, 13B and 13C during therapy planning, each of the volume elements being contained in at least one subvolume element. Overlapping areas 17A and 17B are also shown in FIG. 1. Volume elements inside these overlapping areas 17A and 17B are irradiated during the irradiation of two subvolumes. The splitting up of the particle “sub” numbers into the twofold irradiation during the irradiation of the two subvolumes is performed, for example, in the shape of a ramp (see FIG. 2 for illustration).

Each subvolume 13A, 13B, 13C is assigned a center 19A, 19B, 19C, the respective center coinciding with the isocenter of the scanning system 3 during the irradiation of one of the subvolumes. In FIG. 1, the center 19B of the scanning volume 13B coincides with the isocenter of the scanning system 3. During the irradiation, the patient holding device 21, such as a patient couch in FIG. 1, is moved in such a way that the centers of the subvolumes are positioned at the isocenter of the scanning system 3 one after the other with time.

The splitting up into three subvolumes 33A, 33B, 33C with the centers 35A, 35B, 35C is illustrated in FIG. 2 with a volume 31 illustrated schematically in section. When splitting up the target volume 31, a volume element 37 or a boundary of the target volume 31 may be prescribed, starting from which the splitting up is performed. A size of the overlapping areas 39 may be prescribed.

The right-hand half of FIG. 2 illustrates the irradiation in the Z-direction. The associated distributions of particle “sub” numbers for the three subvolumes 33A, 33B, 33C for a scan in the X-direction are indicated by the lengths of the arrows. In the overlapping areas 39, there is a ramp-type drop in the particle “sub” number distributions (lengths of arrows) toward the edge of the subvolumes 33A and 33B, respectively. As an alternative, it is possible to perceive any type of splitting up of the particle “sub” numbers in the transitional area. Because of the ramp-type formation of the particle “sub” number distributions, the irradiation becomes insensitive to incorrect positioning in the X-direction.

During the irradiation of the various subvolumes, the patient may be displaced at will depending on the position and formation of the volume 31 to be irradiated. For example, a displacement of the patient only in the X-direction takes place in FIG. 2 during the transition from subvolume 33A to subvolume 33B. A displacement in the X- and Y-directions is required in the case of a subsequent alignment of the center 35C with the isocenter. (A displacement of a center in the Z-direction corresponds to a change in the particle energy).

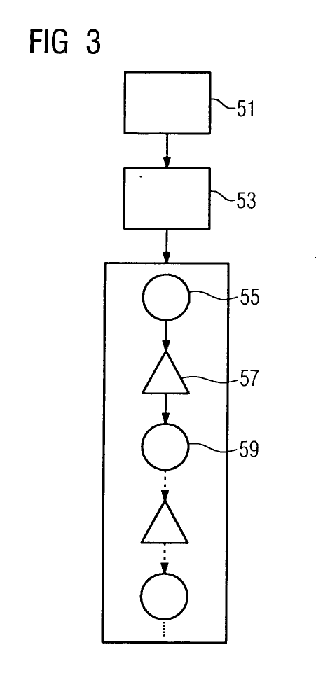

FIG. 3 illustrates an irradiation method having an irradiation sequence in which a number of subvolumes are irradiated. The irradiation precedes a preparatory act 51 in which the patient is positioned and fixed in the appropriate position on a positioning device.

The patient is positioned in front of the scanning system in accordance with the therapy plan in such a way that a center of a first one of the subvolumes coincides with the isocenter of the scanning system. In this position, a verification of position 53 is carried out (for example by imaging methods such as computer tomography), in order to check that the position and alignment of the tissue to be irradiated corresponds to the position and alignment present in the therapy planning.

Once this is confirmed, the first subvolume is irradiated 55. Upon termination of the irradiation 55, a displacement operation 57 of the patient supporting device is driven in such a way that the center of a second one of the subvolumes coincides with the isocenter of the scanning system. The irradiation 59 of the second subvolume is now performed. Depending on the number of subvolumes to be irradiated, the operation of driving the patient couch in order to displace the patient is repeated with the aim of superposing the isocenter of the scanning system on a new center, and the irradiation that follows continues until the volume to be irradiated is irradiated in accordance with the prescribed dose distribution.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

1. A method for determining control parameters of a therapy system for an irradiation sequence of a target volume to be irradiated from an irradiation direction, the target volume comprising a multiplicity of volume elements, each of the volume elements being assigned a particle number, and the target volume being greater than a maximum scanning volume determined by a scanning area of a scanning system of the therapy system, the method comprising:
automatically splitting up the target volume into a number of subvolumes, each of the subvolumes being no greater than the maximum scanning volume, and each of the volume elements being comprised in at least one subvolume,
automatically determining a patient position and/or patient holder position as a first control parameter in which one of the subvolumes is arranged in the scanning area, and
automatically determining a particle “sub” number for each volume element of a subvolume as a second control parameter, such that the sum of all the particle “sub” numbers of a first volume element corresponds to the required particle number of the first volume element.

2. The method as claimed in claim 1, wherein a first one of the subvolumes is arranged in the volume before the automatic splitting up.

3. The method as claimed in claim 1, wherein a size of an overlapping area is prescribed.

4. The method as claimed in claim 3, wherein the overlapping area is displayed on a display unit and/or may be corrected.

5. The method as claimed in claim 3, wherein the splitting up of particle “sub” numbers of a volume element in the overlapping area of two subvolumes, and/or a gradient of a dose ramp, determined by the particle “sub” numbers, is prescribed in the transitional area.

6. The method as claimed in claim 1, wherein a position of the subvolumes is displayed on a display unit.

7. A radiation therapy planning device for generating control parameters of a therapy system for an irradiation sequence on a volume to be irradiated from an irradiation direction, the volume consisting of a multiplicity of volume elements, each of the volume elements being assigned a particle number, and the volume being greater than a maximum scanning volume determined by a scanning area of a scanning system of the therapy system, the radiation therapy planning device being operable to:
automatically split up the volume to be irradiated into a number of subvolumes, each of the subvolumes being no greater than the maximum scanning volume, and each of the volume elements being contained in at least one subvolume,
automatically determining a first control parameter for positioning the subvolumes in the scanning area of the scanning system, and
automatically determine a particle “sub” number for each volume element of a subvolume as a second control parameter, such that the sum of all the particle “sub” numbers of a first volume element corresponds to the particle numbers of the first volume element.

8. An irradiation method for irradiating a patient with high-energy particles from a therapy system, a volume to be irradiated comprising of a multiplicity of volume elements, each of the volume elements being assigned a particle number, and the volume being greater than a maximum scanning volume determined by a scanning area of a scanning system of the therapy system, the method comprising:
irradiating the volume using an irradiation sequence that is based on subvolumes, each of the subvolumes being no greater than the maximum scanning volume, and each of the volume elements being contained in at least one subvolume,
verifying an irradiation position of the patient prior to using the irradiation sequence, and
irradiating the subvolumes one after the other with time positioned in the scanning area and from the same irradiation direction, the volume elements inside the scanning area being irradiated with particle “sub” numbers by driving the scanning system in such a way that the sum of all the particle “sub” numbers of a volume element corresponds to the particle number of this volume element.

9. The irradiation method as claimed in claim 8, comprising: determining control parameters of the therapy system, the control parameters being used to position and irradiate the subvolumes.

10. A particle therapy system for irradiating a volume of a patient that is to be irradiated, the particle therapy system comprising:
a scanning system operable to set a position of a particle beam in two dimensions in the region of a scanning area,
a positioning device operable to position the volume of the patient that is to be irradiated relative to the scanning system, the volume being greater than a maximum scanning volume determined by the scanning area, and
a control unit for driving the scanning system and the positioning device,
wherein the particle therapy system being operable to carry out an irradiation during which subvolumes are one after the other positioned in the scanning area and are irradiated from an irradiation direction, and
wherein the control unit is operable to process control parameters that position the subvolumes in the scanning area of the scanning system, and irradiate a volume element of the subvolume with a particle “sub” number, such that the sum of all the particle “sub” numbers of a volume element corresponds to a planned particle number of this volume element.

11. The particle therapy system as claimed in claim 10, wherein the control unit is operable to carry out an irradiation method.
